(19) United States
(12) Patent Application Publication
(10) Pub. No.: US 2023/0200409 A1
(43) Pub. Date: Jun. 29, 2023

(54) A KIND OF DIET CAPABLE OF ENHANCING THE IMMUNE FUNCTION OF BABY DOGS AND ITS PREPARATION AND USAGE

(71) Applicant: Anhui Science And Technology University, Chuzhou City (CN)

(72) Inventors: Youfang Gu, Chuzhou City (CN); Chongmei Ruan, Chuzhou City (CN); Qianqian Hu, Chuzhou City (CN); Pengfei Ye, Chuzhou City (CN); Deyi Liu, Chuzhou City (CN); Shaojun He, Chuzhou City (CN); Man Ren, Chuzhou City (CN); Yongjie Xiong, Chuzhou City (CN); Zuhuai Ding, Chuzhou City (CN); Xuecheng Song, Chuzhou City (CN)

(21) Appl. No.: 17/257,974

(22) PCT Filed: Aug. 18, 2020
(86) PCT No.: PCT/CN2020/109766

§ 371 Date: Jan. 5, 2021

(30) Foreign Application Priority Data
Dec. 9, 2019 (CN) 2019112482845

(51) Int. Cl. A23K 10/22; A23K 50/40; A23K 10/30; A23K 10/14; A23K 10/18

(52) U.S. Cl. CPC A23K 10/22; A23K 50/40; A23K 10/30; A23K 10/14; A23K 10/18

(57) ABSTRACT

The present invention discloses a kind of diet capable of enhancing the immune function of baby dogs, wherein, the raw materials of which consist of main components, first auxiliary components, and second compounding components; wherein, the main components in terms of mass percent consist of: 80-90% of high protein material, 5-10% of vegetable adhesive, 0.1-0.2% of multiple vitamin, 0.01-0.1% of compound trace elements, and the remain is fermentation preparation; and the first auxiliary components in terms of mass percent consist of: 20-40% of montmorillonite, 10-30% of fish meal, 10-16% of seaweed, and the remain is Chinese herbal medicines; and the second compounding components consist of: beer yeast, linseed oil, cod liver oil, and corn oil. The present invention discloses a method for preparing the aforesaid diet capable of enhancing the immune function of baby dogs. The present invention discloses a method for using the aforesaid diet capable of enhancing the immune function of baby dogs.

### FIELD OF THE INVENTION

The present invention relates to the technical field of animal feeds, particularly to a kind of diet capable of enhancing the immune function of baby dogs and its preparation and usage.

### BACKGROUND OF THE INVENTION

Baby dogs are full of energy, they have relatively fast metabolism with smaller gastric volume. Their nutritional requirements for the same per kilogram of body weight are higher than that of large and medium-sized adult dogs. If such special needs of baby dogs cannot be satisfied, and the baby dogs are artificially caused to be in hungry for a long time, it will seriously affect the growth and health of baby dogs; insufficient intake of baby dogs can lead to alterations in metabolism, hypoglycemia, emaciation, and the fat layers become thin, as well as reduce oxygen consumption and quantity of flow of peripheral circulation, and thus, result in their ability to maintain body temperature is poor; in addition, their immune function will be impaired. Due to the protein synthesis reduced, such immune organs as thymus, lymph nodes, tonsils, and spleen are atrophied, and their body suffer from lack of various immune activators, as well as their cellular and humoral immunity are reduced, and thus, is easy to cause various infections. As for baby dogs, finding an approach to prepare a kind of diet capable of enhancing their immunity and satisfying their nutritional requirements for growth and development as well as keeping healthy has become a technical problem needs to be solved urgently.

### SUMMARY OF THE INVENTION

The purpose of the present invention is to address the shortcomings in the prior art, and propose a kind of diet capable of enhancing the immune function of baby dogs.

A kind of diet capable of enhancing the immune function of baby dogs, wherein, the raw materials of which consist of main components, first auxiliary components, and second compounding components;

The main components in terms of mass percent consist of: 80-90% of high protein material, 5-10% of vegetable adhesive, 0.1-0.2% of multiple vitamins, 0.01-0.1% of compound trace elements, and the remain is fermentation preparation;

The first auxiliary components in terms of mass percent consist of: 20-40% of montmorillonite, 10-30% of fish meal, 10-16% of seaweed, and the remain is Chinese herbal medicines;

The second compounding components consist of: beer yeast, linseed oil, cod liver oil, and corn oil.

Preferably, the mass ratio among the main components, the first auxiliary components, and the second compounding components is 80-90:8-12:4-6.

Preferably, the high-protein materials consist of: chicken, shrimp meal, eggs, bean pulp; and the mass ratio among chicken, shrimp meal, eggs, and bean pulp is 15-25:4-12: 2-8:10-18.

Preferably, the vegetable adhesive is composed of purple sweet potato flour and corn starch, and the mass ratio between purple sweet potato flour and corn starch is 2-6:3-6.

Preferably, the multiple vitamins consist of: vitamin A, vitamin D3, vitamin E, vitamin B1, vitamin B2, nicotinic acid, pantothenic acid, vitamin B6, folic acid, biotin, vitamin B12, and choline chloride.

Preferably, the compound trace elements consist of: sodium chloride, magnesium lactate, ferrous lactate, copper methionine, zinc lactate, manganese sulfate, calcium iodate, and selenium yeast.

Preferably, the fermentation preparation is composed of fermentative bacteria preparation and proteinase preparation, and the mass ratio between the fermentative bacteria preparation and the proteinase preparation is 0.1-0.12:0.1-0.2.

Preferably, the fermentative bacteria preparation consists of: Pichia pastoris, Lactobacillus brucei, Lactobacillus plantarum, Pediococcus acidilactici, and Lactobacillus, and the inoculation amount is 2.5×108 cfu/kg.

Preferably, the Chinese herbal medicines in terms of part by mass consist of: 1-6 parts of Ophiopogon japonicus, 2-10 parts of Astragalus, 2-8 parts of Matsutake, 3-12 parts of Panax notoginseng, 1-5 parts of Sea buckthorn, 2-6 parts of Honeysuckle, 4-12 parts of Atractylodes macrocephala, 10-16 parts of Chinese yam, 1-2 parts of Achyranthes bidentata and 1-2 parts of Poria cocos.

Preferably, the mass ratio among beer yeast, linseed oil, cod liver oil, and corn oil is 10-16:2-4:1-3:1-2.

The method for preparing the aforesaid diet capable of enhancing the immune function of baby dogs consists of following steps:

S1. Mix the high protein materials evenly, and add proteinase preparations for enzymatic hydrolysis, after hyperthermic treatment, add vegetable adhesive, and then stir evenly, after that, mill them, and then add water, and multiple vitamins as well as compound trace elements for stirring evenly, and then, cook them and cool them down, as well as inoculate fermentative bacteria preparation, after that, vacuumize them, and thus, obtain the main component;

S2. Mix the Chinese herbal medicines evenly, and add water, and then, bring to a boil, after that, cook them in the boiling state, and then, filter them, and thus, obtain the first stage filtrate and the filter residue; and then, add water to the filter residue, after bringing to a boil, cook them in the boiling state, and then, filter them, and thus, obtain the second stage filtrate; after that, combine these two filtrates, and then, add montmorillonite and fish meal for stirring and concentrating, after that, pulverize them by pulverizer and sieve them, and then, vacuumize them, and thus, obtain the first auxiliary component;

S3. Mix the beer yeast, linseed oil, cod liver oil, and corn oil evenly, and then, vacuumize them, and thus, obtain the second compounding component.

Preferably, in S1, the enzymolysis temperature is at 30-40° C., and the enzymolysis time is 12-16 h.

Preferably, in S1, the temperature is increased to 85-95° C. for thermal treatment for 20-40 min after enzymolysis.

Preferably, in S1, the milling time is 20-40 min, and the milling pressure is 0.12-0.2 MPa.

Preferably, in S1, the cooking time is 20-40 min.

Preferably, in S2, the temperature is adjusted to 80-90° C. for stirring after adding montmorillonite and fish meal, and then, concentrate them to the water content of 20-40 wt %.

Preferably, in S2, sieve them through a 100-mesh sieve after pulverizing.

The method for using the aforesaid diet capable of enhancing the immune function of baby dogs consists of following steps: heat the main component, and stir it evenly during the heating process, after that, add the first auxiliary component and keep stirring, and then, add the second compounding component and keep stirring, after that, feed them to baby dogs.

Preferably, the heating temperature is at 30-40° C., and the heating time is 10-20 min.

The diet of the present invention is carefully developed according to the main physiological characteristics of baby dogs, which uses natural high-quality chicken, shrimp meal, eggs, with adding natural grains and Chinese herbal medicines to completely satisfy the nutrition and energy required for the rapid growth and development of dogs during the juvenile stage, and promote metabolism. In addition, such diet is formulated into a semi-humid solid state suitable for chewing by baby dogs with vulnerable teeth, and thus, is extremely suitable for providing healthy food to small baby dogs with undeveloped gastrointestinal function.

The present invention is rich in animal albumen and vegetable protein, and is also rich in trace elements such as calcium and phosphorus, in addition, the dog food contains some Chinese herbal medicine components, which greatly enhances the immunity of baby dogs and guarantees baby dogs to grow up healthily irrespective of any environment.

The formula of the present invention is developed according to the modern animal nutrition theory and based upon enhancing the nutritional balance of the diet of baby dogs, as well as develops a diet capable of conditioning baby dog's own immunity of the organism by adjusting the nutritional structure of baby dogs, and cooperating with Chinese herbal medicines having functions of invigorating qi for consolidating superficies, nourishing and reinforcing liver and kidney, strengthening spleen and promoting fluid, which compatibility of medicines takes into account the palatability of the finished product and the needs of nutritional metabolism. By means of the adjustment of the dietary structure and the overall conditioning effect of the Chinese herbal compound prescription, and on the principle of “homology of medicine and food”, such diet not only has comprehensive nutrition and well palatability, but also provides various nutrient substances, such as comprehensive organic trace elements, vitamins, minerals, and amino acids, as well as a variety of highly active digestive enzymes, probiotics, and polypeptides. In addition, it can improve the digestibility and absorptivity of baby dogs to the diet and the palatability of the diet, and thus, significantly improve the immunity of baby dogs, and such diet is free from any preservatives, which is pure natural green food and belongs to a kind of high-end superior-quality diet for baby dogs.

Among the main components of the present invention, chicken, shrimp meal, eggs, and bean pulp are compounded with purple sweet potato flour and corn starch after being enzymatically hydrolyzed by protease, the nutrition of which, especially the nutrition level of amino acid, is superior to the current domestic dog food, and its main nutritional indexes up to the standards of foreign high-quality commercial dog food. Cooperating with the second compounding component, the linoleic acid content of the obtained diet reaches about 5% by testing and the linolenic acid content reaches about 1.2%; in addition, the compound of the main component and the first auxiliary component can improve the health condition of baby dogs significantly, and enhance the immunity obviously. Feeding baby dogs by the present invention makes the skin of baby dogs smoother and more exquisite, moisturized and elastic, and brings their hair glossy, softer and smoother.

As for the Chinese herbal medicines used as the first auxiliary component of the present invention, the Astragalus and the Poria cocos are compounded, which have the effects of invigorating the lack of vitality of the kidneys and conditioning the activity of osteoblasts, and thus, can prevent baby dogs from suffering osteoporosis, lumbar spondylosis and cervical spondylosis associated with rickets. In addition, further adding Ophiopogon japonicus and Achyranthes bidentata can nourish the kidney and marrow and regulate the conversion of vitamin D in the kidney, and its epidermal cortex is wide, so that is conducive to increase the minerals in the diet, and the regulation of the liver function can boost the metabolism of dogs to dietary nutrients of diet, and thus, improves the endocrine function of kidney to promote the activation of vitamin D3 in the kidney, so that regulates the utilization of body to calcium and phosphorus. Moreover, the Atractylodes macrocephala and the Chinese yam have effects of invigorating the spleen and lungs, reinforcing the stomach and kidney, and thus, improve the digestive function of puppies, and regulate the absorption of nutrients, especially the absorption of calcium and phosphorus. Due to baby dogs have delicate sense of smell, the diet cooperates with the concentration of montmorillonite, which can effectively absorb Chinese herbal medicines, and combines with the effect of fish meal, and thus can effectively dilute, even cover the smell of Chinese herbal medicines, so that is easy for baby dogs to eat. After montmorillonite passes through the gastrointestinal tract of baby dogs, the internal Chinese herbal medicine components are digested and absorbed, and the montmorillonite can further boost intestinal tract movement and promote the defecation of baby dogs, as well as further prevent and avoid digestive diseases.

### DETAILED DESCRIPTION OF THE INVENTION

The present invention will be further explained below in conjunction with specific embodiments.

### Embodiment 1

A kind of diet capable of enhancing the immune function of baby dogs, wherein, the raw materials of which consist of 80 kg of the main components, 12 kg of the first auxiliary components, and 4 kg of the second compounding components.

The main components in terms of mass percent consist of: 90% of high protein material, 5% of vegetable adhesive, 0.2% of multiple vitamins, 0.01% of compound trace elements, and the remain is fermentation preparation.

The high-protein materials consist of: 25 kg of chicken, 4 kg of shrimp meal, 8 kg of eggs, 10 kg of bean pulp; the vegetable adhesive is composed of 6 kg of purple sweet potato flour and 3 kg of corn starch; and the fermentation preparation is composed of 0.12 kg of fermentative bacteria preparation and 0.1 kg of proteinase preparation, wherein, the fermentative bacteria preparation consists of: Pichia pastoris, Lactobacillus brucei, Lactobacillus plantarum, Pediococcus acidilactici, and Lactobacillus, and the inoculation amount is 2.5×108 cfu/kg.

The multiple vitamins consist of: vitamin A, vitamin D3, vitamin E, vitamin B1, vitamin B2, nicotinic acid, pantothenic acid, vitamin B6, folic acid, biotin, vitamin B12, and choline chloride; and the compound trace elements consist of: sodium chloride, magnesium lactate, ferrous lactate, copper methionine, zinc lactate, manganese sulfate, calcium iodate, and selenium yeast.

The contents of vitamins and trace elements are as follows:

| | | |
|---|---|---|
| Vitamin A | 5779.35 | IU/kg |
| Vitamin D₃ | 613.22 | IU/kg |
| Vitamin E | 57.38 | IU/kg |
| Vitamin B₁ | 1.23 | mg/kg |
| Vitamin B₂ | 2.55 | mg/kg |
| Nicotinic acid | 12.93 | mg/kg |
| Pantothenic acid | 11.70 | mg/kg |
| Vitamin B₆ | 1.23 | mg/kg |
| Folic acid | 231.17 | μg/kg |
| Biotin | 113.4 | μg/kg |
| Vitamin B₁₂ | 27.87 | μg/kg |
| Choline | 1.34 | mg/kg |
| Sodium chloride | 12.38 | g/kg |
| Magnesium | 450.07 | mg/kg |
| Iron | 67.51 | mg/kg |
| Copper | 8.18 | mg/kg |
| Zinc | 56.26 | mg/kg |
| Manganese | 5.63 | mg/kg |
| Iodine | 1.74 | mg/kg |
| Selenium | 0.96 | mg/kg |

The first auxiliary components in terms of mass percent consist of: 40% of montmorillonite, 10% of fish meal, 16% of seaweed, and the remain is Chinese herbal medicines; wherein, the Chinese herbal medicines consist of: 1 kg of Ophiopogon japonicus, 10 kg of Astragalus, 2 kg of Matsutake, 12 kg of Panax notoginseng, 1 kg of Sea buckthorn, 6 kg of Honeysuckle, 4 kg of Atractylodes macrocephala, 16 kg of Chinese yam, 1 kg of Achyranthes bidentata and 2 kg of Poria cocos.

The second compounding components consist of: 10 kg of beer yeast, 4 kg of linseed oil, 1 kg of cod liver oil, and 2 kg of corn oil.

The method for preparing the aforesaid diet capable of enhancing the immune function of baby dogs consists of following steps:

S1. Mix the high protein materials evenly, and add proteinase preparations for enzymatic hydrolysis for 12 h at the temperature of 30° C., and then, heat up to 95° C. and complete hyperthermic treatment for 20 min, after that, feed them to the blender mixer, and then, add vegetable adhesive and stir evenly, after that, feed them to the mill and grind it for 40 min, and the milling pressure is 0.12 MPa, then, add water till the water content reaches 60 wt %, after that, add multiple vitamins and compound trace elements, stir them evenly, and then, cook them for 20 min, and cool them down, as well as inoculate fermentative bacteria preparation, after that, vacuumize them, and thus, obtain the main component;

S2. Mix the Chinese herbal medicines evenly, and then, feed them to extracting tank, and add water of 3 times of the total mass of the Chinese herbal medicines, and then, bring to a boil, after that, cook them in the boiling state for 2 h, and then, filter them, and thus, obtain the first stage filtrate and the filter residue; and then, add water of 2 times of the total mass of the filter residue to the filter residue, after bringing to a boil, cook them in the boiling state for 2 h, and then, filter them, and thus, obtain the second stage filtrate; after that, combine these two filtrates, and then, add montmorillonite and fish meal for stirring at the temperature of 90° C., and then, concentrate them till the water content reaches 20 wt %, and then sieve them through a 100-mesh sieve after pulverizing by pulverizer, and then, vacuumize them, and thus, obtain the first auxiliary component;

S3. Mix the beer yeast, linseed oil, cod liver oil, and corn oil evenly, and then, vacuumize them, and thus, obtain the second compounding component.

The method for using the aforesaid diet capable of enhancing the immune function of baby dogs consists of following steps: heat the main component for 10 min at the temperature of 40° C., and stir them evenly during the heating process, after that, add the first auxiliary component and keep stirring for 15 min, and then, add the second compounding component and keep stirring for 10 min, after that, feed them to baby dogs.

### Embodiment 2

A kind of diet capable of enhancing the immune function of baby dogs, wherein, the raw materials of which consist of 90 kg of the main components, 8 kg of the first auxiliary components, and 6 kg of the second compounding components.

The main components in terms of mass percent consist of: 80% of high protein material, 10% of vegetable adhesive, 0.1% of multiple vitamins, 0.1% of compound trace elements, and the remain is fermentation preparation.

The high-protein materials consist of: 15 kg of chicken, 12 kg of shrimp meal, 2 kg of eggs, 18 kg of bean pulp; the vegetable adhesive is composed of 2 kg of purple sweet potato flour and 6 kg of corn starch; and the fermentation preparation is composed of 0.1 kg of fermentative bacteria preparation and 0.2 kg of proteinase preparation, wherein, the fermentative bacteria preparation consists of: Pichia pastoris, Lactobacillus brucei, Lactobacillus plantarum, Pediococcus acidilactici, and Lactobacillus, and the inoculation amount is 2.5×108 cfu/kg.

The multiple vitamins consist of: vitamin A, vitamin D3, vitamin E, vitamin B1, vitamin B2, nicotinic acid, pantothenic acid, vitamin B6, folic acid, biotin, vitamin B12, and choline chloride; and the compound trace elements consist of: sodium chloride, magnesium lactate, ferrous lactate, copper methionine, zinc lactate, manganese sulfate, calcium iodate, and selenium yeast.

The contents of vitamins and trace elements are as follows:

| | | |
|---|---|---|
| Vitamin A | 5779.35 | IU/kg |
| Vitamin D₃ | 613.22 | IU/kg |
| Vitamin E | 57.38 | IU/kg |
| Vitamin B₁ | 1.23 | mg/kg |
| Vitamin B₂ | 2.55 | mg/kg |
| Nicotinic acid | 12.93 | mg/kg |
| Pantothenic acid | 11.70 | mg/kg |
| Vitamin B₆ | 1.23 | mg/kg |
| Folic acid | 231.17 | μg/kg |
| Biotin | 113.4 | μg/kg |
| Vitamin B₁₂ | 27.87 | μg/kg |
| Choline | 1.34 | mg/kg |
| Sodium chloride | 12.38 | g/kg |
| Magnesium | 450.07 | mg/kg |
| Iron | 67.51 | mg/kg |
| Copper | 8.18 | mg/kg |
| Zinc | 56.26 | mg/kg |
| Manganese | 5.63 | mg/kg |
| Iodine | 1.74 | mg/kg |
| Selenium | 0.96 | mg/kg |

The first auxiliary components in terms of mass percent consist of: 20% of montmorillonite, 30% of fish meal, 10% of seaweed, and the remain is Chinese herbal medicines; wherein, the Chinese herbal medicines consist of: 6 kg of Ophiopogon japonicus, 2 kg of Astragalus, 8 kg of Matsutake, 3 kg of Panax notoginseng, 5 kg of Sea buckthorn, 2 kg of Honeysuckle, 12 kg of Atractylodes macrocephala, 10 kg of Chinese yam, 2 kg of Achyranthes bidentata and 1 kg of Poria cocos.

The second compounding components consist of: 16 kg of beer yeast, 2 kg of linseed oil, 3 kg of cod liver oil, and 1 kg of corn oil.

The method for preparing the aforesaid diet capable of enhancing the immune function of baby dogs consists of following steps:

S1. Mix the high protein materials evenly, and add proteinase preparations for enzymatic hydrolysis for 16 h at the temperature of 40° C., and then, heat up to 85° C. and complete hyperthermic treatment for 40 min, after that, feed them to the blender mixer, and then, add vegetable adhesive and stir evenly, after that, feed them to the mill and grind it for 20 min, and the milling pressure is 0.2 MPa, then, add water till the water content reaches 40 wt %, after that, add multiple vitamins and compound trace elements, stir them evenly, and then, cook them for 40 min, and cool them down, as well as inoculate fermentative bacteria preparation, after that, vacuumize them, and thus, obtain the main component;

S2. Mix the Chinese herbal medicines evenly, and then, feed them to extracting tank, and add water of 2 times of the total mass of the Chinese herbal medicines, and then, bring to a boil, after that, cook them in the boiling state for 4 h, and then, filter them, and thus, obtain the first stage filtrate and the filter residue; and then, add water of 1.5 times of the total mass of the filter residue to the filter residue, after bringing to a boil, cook them in the boiling state for 4 h, and then, filter them, and thus, obtain the second stage filtrate; after that, combine these two filtrates, and then, add montmorillonite and fish meal for stirring at the temperature of 80° C., and then, concentrate them till the water content reaches 40 wt %, and then sieve them through a 100-mesh sieve after pulverizing by pulverizer, and then, vacuumize them, and thus, obtain the first auxiliary component;

S3. Mix the beer yeast, linseed oil, cod liver oil, and corn oil evenly, and then, vacuumize them, and thus, obtain the second compounding component.

The method for using the aforesaid diet capable of enhancing the immune function of baby dogs consists of following steps: heat the main component for 20 min at the temperature of 30° C., and stir them evenly during the heating process, after that, add the first auxiliary component and keep stirring for 5 min, and then, add the second compounding component and keep stirring for 20 min, after that, feed them to baby dogs.

### Embodiment 3

A kind of diet capable of enhancing the immune function of baby dogs, wherein, the raw materials of which consist of 82 kg of the main components, 11 kg of the first auxiliary components, and 4.5 kg of the second compounding components.

The main components in terms of mass percent consist of: 87% of high protein material, 6% of vegetable adhesive, 0.18% of multiple vitamins, 0.02% of compound trace elements, and the remain is fermentation preparation.

The high-protein materials consist of: 22 kg of chicken, 6 kg of shrimp meal, 6 kg of eggs, 12 kg of bean pulp; the vegetable adhesive is composed of 5 kg of purple sweet potato flour and 4 kg of corn starch; and the fermentation preparation is composed of 0.115 kg of fermentative bacteria preparation and 0.12 kg of proteinase preparation, wherein, the fermentative bacteria preparation consists of: Pichia pastoris, Lactobacillus brucei, Lactobacillus plantarum, Pediococcus acidilactici, and Lactobacillus, and the inoculation amount is 2.5×108 cfu/kg.

The multiple vitamins consist of: vitamin A, vitamin D3, vitamin E, vitamin B1, vitamin B2, nicotinic acid, pantothenic acid, vitamin B6, folic acid, biotin, vitamin B12, and choline chloride; and the compound trace elements consist of: sodium chloride, magnesium lactate, ferrous lactate, copper methionine, zinc lactate, manganese sulfate, calcium iodate, and selenium yeast.

The contents of vitamins and trace elements are as follows:

| | | |
|---|---|---|
| Vitamin A | 5779.35 | IU/kg |
| Vitamin D₃ | 613.22 | IU/kg |
| Vitamin E | 57.38 | IU/kg |
| Vitamin B₁ | 1.23 | mg/kg |
| Vitamin B₂ | 2.55 | mg/kg |
| Nicotinic acid | 12.93 | mg/kg |
| Pantothenic acid | 11.70 | mg/kg |
| Vitamin B₆ | 1.23 | mg/kg |
| Folic acid | 231.17 | μg/kg |
| Biotin | 113.4 | μg/kg |
| Vitamin B₁₂ | 27.87 | μg/kg |
| Choline | 1.34 | mg/kg |
| Sodium chloride | 12.38 | g/kg |
| Magnesium | 450.07 | mg/kg |
| Iron | 67.51 | mg/kg |
| Copper | 8.18 | mg/kg |
| Zinc | 56.26 | mg/kg |
| Manganese | 5.63 | mg/kg |
| Iodine | 1.74 | mg/kg |
| Selenium | 0.96 | mg/kg |

The first auxiliary components in terms of mass percent consist of: 35% of montmorillonite, 15% of fish meal, 14% of seaweed, and the remain is Chinese herbal medicines; wherein, the Chinese herbal medicines consist of: 2 kg of Ophiopogon japonicus, 8 kg of Astragalus, 4 kg of Matsutake, 10 kg of Panax notoginseng, 2 kg of Sea buckthorn, 5 kg of Honeysuckle, 6 kg of Atractylodes macrocephala, 14 kg of Chinese yam, 1.3 kg of Achyranthes bidentata and 1.8 kg of Poria cocos.

The second compounding components consist of: 12 kg of beer yeast, 3.5 kg of linseed oil, 1.5 kg of cod liver oil, and 1.7 kg of corn oil.

The method for preparing the aforesaid diet capable of enhancing the immune function of baby dogs consists of following steps:

S1. Mix the high protein materials evenly, and add proteinase preparations for enzymatic hydrolysis for 15 h at the temperature of 32° C., and then, heat up to 88° C. and complete hyperthermic treatment for 35 min, after that, feed them to the blender mixer, and then, add vegetable adhesive and stir evenly, after that, feed them to the mill and grind it for 25 min, and the milling pressure is 0.18 MPa, then, add water till the water content reaches 45 wt %, after that, add multiple vitamins and compound trace elements, stir them evenly, and then, cook them for 35 min, and cool them down, as well as inoculate fermentative bacteria preparation, after that, vacuumize them, and thus, obtain the main component;

S2. Mix the Chinese herbal medicines evenly, and then, feed them to extracting tank, and add water of 2.2 times of the total mass of the Chinese herbal medicines, and then, bring to a boil, after that, cook them in the boiling state for 3.5 h, and then, filter them, and thus, obtain the first stage filtrate and the filter residue; and then, add water of 1.6 times of the total mass of the filter residue to the filter residue, after bringing to a boil, cook them in the boiling state for 3.5 h, and then, filter them, and thus, obtain the second stage filtrate; after that, combine these two filtrates, and then, add montmorillonite and fish meal for stirring at the temperature of 82° C., and then, concentrate them till the water content reaches 35 wt %, and then sieve them through a 100-mesh sieve after pulverizing by pulverizer, and then, vacuumize them, and thus, obtain the first auxiliary component;

S3. Mix the beer yeast, linseed oil, cod liver oil, and corn oil evenly, and then, vacuumize them, and thus, obtain the second compounding component.

The method for using the aforesaid diet capable of enhancing the immune function of baby dogs consists of following steps: heat the main component for 18 min at the temperature of 33° C., and stir them evenly during the heating process, after that, add the first auxiliary component and keep stirring for 8 min, and then, add the second compounding component and keep stirring for 18 min, after that, feed them to baby dogs.

### Embodiment 4

A kind of diet capable of enhancing the immune function of baby dogs, wherein, the raw materials of which consist of 88 kg of the main components, 9 kg of the first auxiliary components, and 5.5 kg of the second compounding components.

The main components in terms of mass percent consist of: 82% of high protein material, 9% of vegetable adhesive, 0.12% of multiple vitamins, 0.08% of compound trace elements, and the remain is fermentation preparation.

The high-protein materials consist of: 18 kg of chicken, 10 kg of shrimp meal, 4 kg of eggs, 16 kg of bean pulp; the vegetable adhesive is composed of 3 kg of purple sweet potato flour and 5 kg of corn starch; and the fermentation preparation is composed of 0.105 kg of fermentative bacteria preparation and 0.18 kg of proteinase preparation, wherein, the fermentative bacteria preparation consists of: Pichia pastoris, Lactobacillus brucei, Lactobacillus plantarum, Pediococcus acidilactici, and Lactobacillus, and the inoculation amount is 2.5×108 cfu/kg.

The multiple vitamins consist of: vitamin A, vitamin D3, vitamin E, vitamin B1, vitamin B2, nicotinic acid, pantothenic acid, vitamin B6, folic acid, biotin, vitamin B12, and choline chloride; and the compound trace elements consist of: sodium chloride, magnesium lactate, ferrous lactate, copper methionine, zinc lactate, manganese sulfate, calcium iodate, and selenium yeast.

The contents of vitamins and trace elements are as follows:

| | | |
|---|---|---|
| Vitamin A | 5779.35 | IU/kg |
| Vitamin D₃ | 613.22 | IU/kg |
| Vitamin E | 57.38 | IU/kg |
| Vitamin B₁ | 1.23 | mg/kg |
| Vitamin B₂ | 2.55 | mg/kg |
| Nicotinic acid | 12.93 | mg/kg |
| Pantothenic acid | 11.70 | mg/kg |
| Vitamin B₆ | 1.23 | mg/kg |
| Folic acid | 231.17 | μg/kg |
| Biotin | 113.4 | μg/kg |
| Vitamin B₁₂ | 27.87 | μg/kg |
| Choline | 1.34 | mg/kg |
| Sodium chloride | 12.38 | g/kg |
| Magnesium | 450.07 | mg/kg |
| Iron | 67.51 | mg/kg |
| Copper | 8.18 | mg/kg |
| Zinc | 56.26 | mg/kg |
| Manganese | 5.63 | mg/kg |
| Iodine | 1.74 | mg/kg |
| Selenium | 0.96 | mg/kg |

The first auxiliary components in terms of mass percent consist of: 25% of montmorillonite, 25% of fish meal, 12% of seaweed, and the remain is Chinese herbal medicines; wherein, the Chinese herbal medicines consist of: 4 kg of Ophiopogon japonicus, 4 kg of Astragalus, 6 kg of Matsutake, 6 kg of Panax notoginseng, 4 kg of Sea buckthorn, 3 kg of Honeysuckle, 10 kg of Atractylodes macrocephala, 12 kg of Chinese yam, 1.7 kg of Achyranthes bidentata and 1.4 kg of Poria cocos.

The second compounding components consist of: 14 kg of beer yeast, 2.5 kg of linseed oil, 2.5 kg of cod liver oil, and 1.3 kg of corn oil.

The method for preparing the aforesaid diet capable of enhancing the immune function of baby dogs consists of following steps:

S1. Mix the high protein materials evenly, and add proteinase preparations for enzymatic hydrolysis for 13 h at the temperature of 36° C., and then, heat up to 92° C. and complete hyperthermic treatment for 25 min, after that, feed them to the blender mixer, and then, add vegetable adhesive and stir evenly, after that, feed them to the mill and grind it for 35 min, and the milling pressure is 0.14 MPa, then, add water till the water content reaches 55 wt %, after that, add multiple vitamins and compound trace elements, stir them evenly, and then, cook them for 25 min, and cool them down, as well as inoculate fermentative bacteria preparation, after that, vacuumize them, and thus, obtain the main component;

S2. Mix the Chinese herbal medicines evenly, and then, feed them to extracting tank, and add water of 2.8 times of the total mass of the Chinese herbal medicines, and then, bring to a boil, after that, cook them in the boiling state for 2.5 h, and then, filter them, and thus, obtain the first stage filtrate and the filter residue; and then, add water of 1.8 times of the total mass of the filter residue to the filter residue, after bringing to a boil, cook them in the boiling state for 2.5 h, and then, filter them, and thus, obtain the second stage filtrate; after that, combine these two filtrates, and then, add montmorillonite and fish meal for stirring at the temperature of 88° C., and then, concentrate them till the water content reaches 25 wt %, and then sieve them through a 100-mesh sieve after pulverizing by pulverizer, and then, vacuumize them, and thus, obtain the first auxiliary component;

S3. Mix the beer yeast, linseed oil, cod liver oil, and corn oil evenly, and then, vacuumize them, and thus, obtain the second compounding component.

The method for using the aforesaid diet capable of enhancing the immune function of baby dogs consists of following steps: heat the main component for 12 min at the temperature of 37° C., and stir them evenly during the heating process, after that, add the first auxiliary component and keep stirring for 12 min, and then, add the second compounding component and keep stirring for 12 min, after that, feed them to baby dogs.

### Embodiment 5

A kind of diet capable of enhancing the immune function of baby dogs, wherein, the raw materials of which consist of 85 kg of the main components, 10 kg of the first auxiliary components, and 5 kg of the second compounding components.

The main components in terms of mass percent consist of: 85% of high protein material, 7% of vegetable adhesive, 0.15% of multiple vitamins, 0.05% of compound trace elements, and the remain is fermentation preparation.

The high-protein materials consist of: 20 kg of chicken, 8 kg of shrimp meal, 5 kg of eggs, 14 kg of bean pulp; the vegetable adhesive is composed of 4 kg of purple sweet potato flour and 4.5 kg of corn starch; and the fermentation preparation is composed of 0.11 kg of fermentative bacteria preparation and 0.15 kg of proteinase preparation, wherein, the fermentative bacteria preparation consists of: Pichia pastoris, Lactobacillus brucei, Lactobacillus plantarum, Pediococcus acidilactici, and Lactobacillus, and the inoculation amount is 2.5×108 cfu/kg.

The multiple vitamins consist of: vitamin A, vitamin D3, vitamin E, vitamin B1, vitamin B2, nicotinic acid, pantothenic acid, vitamin B6, folic acid, biotin, vitamin B12, and choline chloride; and the compound trace elements consist of: sodium chloride, magnesium lactate, ferrous lactate, copper methionine, zinc lactate, manganese sulfate, calcium iodate, and selenium yeast.

The contents of vitamins and trace elements are as follows:

| | | |
|---|---|---|
| Vitamin A | 5779.35 | IU/kg |
| Vitamin D₃ | 613.22 | IU/kg |
| Vitamin E | 57.38 | IU/kg |
| Vitamin B₁ | 1.23 | mg/kg |
| Vitamin B₂ | 2.55 | mg/kg |
| Nicotinic acid | 12.93 | mg/kg |
| Pantothenic acid | 11.70 | mg/kg |
| Vitamin B₆ | 1.23 | mg/kg |
| Folic acid | 231.17 | μg/kg |
| Biotin | 113.4 | μg/kg |
| Vitamin B₁₂ | 27.87 | μg/kg |
| Choline | 1.34 | mg/kg |
| Sodium chloride | 12.38 | g/kg |
| Magnesium | 450.07 | mg/kg |
| Iron | 67.51 | mg/kg |
| Copper | 8.18 | mg/kg |
| Zinc | 56.26 | mg/kg |
| Manganese | 5.63 | mg/kg |
| Iodine | 1.74 | mg/kg |
| Selenium | 0.96 | mg/kg |

The first auxiliary components in terms of mass percent consist of: 30% of montmorillonite, 20% of fish meal, 13% of seaweed, and the remain is Chinese herbal medicines; wherein, the Chinese herbal medicines consist of: 3 kg of Ophiopogon japonicus, 6 kg of Astragalus, 5 kg of Matsutake, 8 kg of Panax notoginseng, 3 kg of Sea buckthorn, 4 kg of Honeysuckle, 8 kg of Atractylodes macrocephala, 13 kg of Chinese yam, 1.5 kg of Achyranthes bidentata and 1.6 kg of Poria cocos.

The second compounding components consist of: 13 kg of beer yeast, 3 kg of linseed oil, 2 kg of cod liver oil, and 1.5 kg of corn oil.

The method for preparing the aforesaid diet capable of enhancing the immune function of baby dogs consists of following steps:

S1. Mix the high protein materials evenly, and add proteinase preparations for enzymatic hydrolysis for 14 h at the temperature of 34° C., and then, heat up to 90° C. and complete hyperthermic treatment for 30 min, after that, feed them to the blender mixer, and then, add vegetable adhesive and stir evenly, after that, feed them to the mill and grind it for 30 min, and the milling pressure is 0.16 MPa, then, add water till the water content reaches 50 wt %, after that, add multiple vitamins and compound trace elements, stir them evenly, and then, cook them for 30 min, and cool them down, as well as inoculate fermentative bacteria preparation, after that, vacuumize them, and thus, obtain the main component;

S2. Mix the Chinese herbal medicines evenly, and then, feed them to extracting tank, and add water of 2.5 times of the total mass of the Chinese herbal medicines, and then, bring to a boil, after that, cook them in the boiling state for 3 h, and then, filter them, and thus, obtain the first stage filtrate and the filter residue; and then, add water of 1.7 times of the total mass of the filter residue to the filter residue, after bringing to a boil, cook them in the boiling state for 3 h, and then, filter them, and thus, obtain the second stage filtrate; after that, combine these two filtrates, and then, add montmorillonite and fish meal for stirring at the temperature of 82-88° C., and then, concentrate them till the water content reaches 30 wt %, and then sieve them through a 100-mesh sieve after pulverizing by pulverizer, and then, vacuumize them, and thus, obtain the first auxiliary component;

S3. Mix the beer yeast, linseed oil, cod liver oil, and corn oil evenly, and then, vacuumize them, and thus, obtain the second compounding component.

The method for using the aforesaid diet capable of enhancing the immune function of baby dogs consists of following steps: heat the main component for 15 min at the temperature of 35° C., and stir them evenly during the heating process, after that, add the first auxiliary component and keep stirring for 10 min, and then, add the second compounding component and keep stirring for 15 min, after that, feed them to baby dogs.

Based upon the analysis of the diet obtained in this embodiment, the total energy of which is 16.36 MJ/kg, and the content of crude protein is 30.74%, the content of crude fat is 6.0%, the content of crude fiber is 7.01%, the calcium content is 1.73%, and phosphorus content is 1.01%.

### Comparative Example 1

Use the wet food made of chicken which purchased from JDcom and is available for ordinary baby dogs.

### Comparative Example 2

A kind of diet for baby dogs, wherein, the raw materials of which consist of 85 kg of the main components and 5 kg of the second compounding components.

The main components in terms of mass percent consist of: 85% of high protein material, 7% of vegetable adhesive, 0.15% of multiple vitamins, 0.05% of compound trace elements, and the remain is fermentation preparation.

The high-protein materials consist of: 20 kg of chicken, 8 kg of shrimp meal, 5 kg of eggs, 14 kg of bean pulp; the vegetable adhesive is composed of 4 kg of purple sweet potato flour and 4.5 kg of corn starch; and the fermentation preparation is composed of 0.11 kg of fermentative bacteria preparation and 0.15 kg of proteinase preparation, wherein, the fermentative bacteria preparation consists of: Pichia pastoris, Lactobacillus brucei, Lactobacillus plantarum, Pediococcus acidilactici, and Lactobacillus, and the inoculation amount is 2.5×108 cfu/kg.

The multiple vitamins consist of: vitamin A, vitamin D3, vitamin E, vitamin B1, vitamin B2, nicotinic acid, pantothenic acid, vitamin B6, folic acid, biotin, vitamin B12, and choline chloride; and the compound trace elements consist of: sodium chloride, magnesium lactate, ferrous lactate, copper methionine, zinc lactate, manganese sulfate, calcium iodate, and selenium yeast.

The contents of vitamins and trace elements are as follows:

| | | |
|---|---|---|
| Vitamin A | 5779.35 | IU/kg |
| Vitamin D₃ | 613.22 | IU/kg |
| Vitamin E | 57.38 | IU/kg |
| Vitamin B₁ | 1.23 | mg/kg |
| Vitamin B₂ | 2.55 | mg/kg |
| Nicotinic acid | 12.93 | mg/kg |
| Pantothenic acid | 11.70 | mg/kg |
| Vitamin B₆ | 1.23 | mg/kg |
| Folic acid | 231.17 | μg/kg |
| Biotin | 113.4 | μg/kg |
| Vitamin B₁₂ | 27.87 | μg/kg |
| Choline | 1.34 | mg/kg |
| Sodium chloride | 12.38 | g/kg |
| Magnesium | 450.07 | mg/kg |
| Iron | 67.51 | mg/kg |
| Copper | 8.18 | mg/kg |
| Zinc | 56.26 | mg/kg |
| Manganese | 5.63 | mg/kg |
| Iodine | 1.74 | mg/kg |
| Selenium | 0.96 | mg/kg |

The second compounding components consist of: 13 kg of beer yeast, 3 kg of linseed oil, 2 kg of cod liver oil, and 1.5 kg of corn oil.

The method for preparing the aforesaid diet for baby dogs consists of following steps:

S1. Mix the high protein materials evenly, and add proteinase preparations for enzymatic hydrolysis for 14 h at the temperature of 34° C., and then, heat up to 90° C. and complete hyperthermic treatment for 30 min, after that, feed them to the blender mixer, and then, add vegetable adhesive and stir evenly, after that, feed them to the mill and grind it for 30 min, and the milling pressure is 0.16 MPa, then, add water till the water content reaches 50 wt %, after that, add multiple vitamins and compound trace elements, stir them evenly, and then, cook them for 30 min, and cool them down, as well as inoculate fermentative bacteria preparation, after that, vacuumize them, and thus, obtain the main component;

S2. Mix the beer yeast, linseed oil, cod liver oil, and corn oil evenly, and then, vacuumize them, and thus, obtain the second compounding component.

The method for using the aforesaid diet for baby dogs consists of following steps: heat the main component for 15 min at the temperature of 35° C., and stir them evenly during the heating process, after that, add the second compounding component and keep stirring for 15 min, and then, feed them to baby dogs.

### Comparative Example 3

A kind of diet for baby dogs, wherein, the raw materials of which consist of 85 kg of the main components and 10 kg of the first auxiliary components.

The main components in terms of mass percent consist of: 85% of high protein material, 7% of vegetable adhesive, 0.15% of multiple vitamins, 0.05% of compound trace elements, and the remain is fermentation preparation.

The high-protein materials consist of: 20 kg of chicken, 8 kg of shrimp meal, 5 kg of eggs, 14 kg of bean pulp; the vegetable adhesive is composed of 4 kg of purple sweet potato flour and 4.5 kg of corn starch; and the fermentation preparation is composed of 0.11 kg of fermentative bacteria preparation and 0.15 kg of proteinase preparation, wherein, the fermentative bacteria preparation consists of: Pichia pastoris, Lactobacillus brucei, Lactobacillus plantarum, Pediococcus acidilactici, and Lactobacillus, and the inoculation amount is 2.5×108 cfu/kg.

The multiple vitamins consist of: vitamin A, vitamin D3, vitamin E, vitamin B1, vitamin B2, nicotinic acid, pantothenic acid, vitamin B6, folic acid, biotin, vitamin B12, and choline chloride; and the compound trace elements consist of: sodium chloride, magnesium lactate, ferrous lactate, copper methionine, zinc lactate, manganese sulfate, calcium iodate, and selenium yeast.

The contents of vitamins and trace elements are as follows:

| | | |
|---|---|---|
| Vitamin A | 5779.35 | IU/kg |
| Vitamin D₃ | 613.22 | IU/kg |
| Vitamin E | 57.38 | IU/kg |
| Vitamin B₁ | 1.23 | mg/kg |
| Vitamin B₂ | 2.55 | mg/kg |
| Nicotinic acid | 12.93 | mg/kg |
| Pantothenic acid | 11.70 | mg/kg |
| Vitamin B₆ | 1.23 | mg/kg |
| Folic acid | 231.17 | μg/kg |
| Biotin | 113.4 | μg/kg |
| Vitamin B₁₂ | 27.87 | μg/kg |
| Choline | 1.34 | mg/kg |
| Sodium chloride | 12.38 | g/kg |
| Magnesium | 450.07 | mg/kg |
| Iron | 67.51 | mg/kg |
| Copper | 8.18 | mg/kg |
| Zinc | 56.26 | mg/kg |
| Manganese | 5.63 | mg/kg |
| Iodine | 1.74 | mg/kg |
| Selenium | 0.96 | mg/kg |

The first auxiliary components in terms of mass percent consist of: 30% of montmorillonite, 20% of fish meal, 13% of seaweed, and the remain is Chinese herbal medicines; wherein, the Chinese herbal medicines consist of: 3 kg of Ophiopogon japonicus, 6 kg of Astragalus, 5 kg of Matsutake, 8 kg of Panax notoginseng, 3 kg of Sea buckthorn, 4 kg of Honeysuckle, 8 kg of Atractylodes macrocephala, 13 kg of Chinese yam, 1.5 kg of Achyranthes bidentata and 1.6 kg of Poria cocos.

The method for preparing the aforesaid diet for baby dogs consists of following steps:

S1. Mix the high protein materials evenly, and add proteinase preparations for enzymatic hydrolysis for 14 h at the temperature of 34° C., and then, heat up to 90° C. and complete hyperthermic treatment for 30 min, after that, feed them to the blender mixer, and then, add vegetable adhesive and stir evenly, after that, feed them to the mill and grind it for 30 min, and the milling pressure is 0.16 MPa, then, add water till the water content reaches 50 wt %, after that, add multiple vitamins and compound trace elements, stir them evenly, and then, cook them for 30 min, and cool them down, as well as inoculate fermentative bacteria preparation, after that, vacuumize them, and thus, obtain the main component;

S2. Mix the Chinese herbal medicines evenly, and then, feed them to extracting tank, and add water of 2.5 times of the total mass of the Chinese herbal medicines, and then, bring to a boil, after that, cook them in the boiling state for 3 h, and then, filter them, and thus, obtain the first stage filtrate and the filter residue; and then, add water of 1.7 times of the total mass of the filter residue to the filter residue, after bringing to a boil, cook them in the boiling state for 3 h, and then, filter them, and thus, obtain the second stage filtrate; after that, combine these two filtrates, and then, add montmorillonite and fish meal for stirring at the temperature of 82-88° C., and then, concentrate them till the water content reaches 30 wt %, and then sieve them through a 100-mesh sieve after pulverizing by pulverizer, and then, vacuumize them, and thus, obtain the first auxiliary component;

The method for using the aforesaid diet for baby dogs consists of following steps: heat the main component for 15 min at the temperature of 35° C., and stir them evenly during the heating process, after that, add the second compounding component and keep stirring for 10 min, and then, feed them to baby dogs.

### Comparative Example 4

A kind of diet for baby dogs, wherein, the raw materials of which consist of 85 kg of the main components, 10 kg of the first auxiliary components, and 5 kg of the second compounding components.

The main components in terms of mass percent consist of: 85% of high protein material, 7% of vegetable adhesive, 0.15% of multiple vitamins, 0.05% of compound trace elements, and the remain is fermentation preparation.

The high-protein materials consist of: 20 kg of chicken, 8 kg of shrimp meal, 5 kg of eggs, 14 kg of bean pulp; the vegetable adhesive is composed of 4 kg of purple sweet potato flour and 4.5 kg of corn starch; and the fermentative bacteria preparation consists of: Pichia pastoris, Lactobacillus brucei, Lactobacillus plantarum, Pediococcus acidilactici, and Lactobacillus, and the inoculation amount is 2.5×108 cfu/kg.

The multiple vitamins consist of: vitamin A, vitamin D3, vitamin E, vitamin B1, vitamin B2, nicotinic acid, pantothenic acid, vitamin B6, folic acid, biotin, vitamin B12, and choline chloride; and the compound trace elements consist of: sodium chloride, magnesium lactate, ferrous lactate, copper methionine, zinc lactate, manganese sulfate, calcium iodate, and selenium yeast.

The contents of vitamins and trace elements are as follows:

| | | |
|---|---|---|
| Vitamin A | 5779.35 | IU/kg |
| Vitamin D₃ | 613.22 | IU/kg |
| Vitamin E | 57.38 | IU/kg |
| Vitamin B₁ | 1.23 | mg/kg |
| Vitamin B₂ | 2.55 | mg/kg |
| Nicotinic acid | 12.93 | mg/kg |
| Pantothenic acid | 11.70 | mg/kg |
| Vitamin B₆ | 1.23 | mg/kg |
| Folic acid | 231.17 | μg/kg |
| Biotin | 113.4 | μg/kg |
| Vitamin B₁₂ | 27.87 | μg/kg |
| Choline | 1.34 | mg/kg |
| Sodium chloride | 12.38 | g/kg |
| Magnesium | 450.07 | mg/kg |
| Iron | 67.51 | mg/kg |
| Copper | 8.18 | mg/kg |
| Zinc | 56.26 | mg/kg |
| Manganese | 5.63 | mg/kg |
| Iodine | 1.74 | mg/kg |
| Selenium | 0.96 | mg/kg |

The first auxiliary components in terms of mass percent consist of: 30% of montmorillonite, 20% of fish meal, 13% of seaweed, and the remain is Chinese herbal medicines; wherein, the Chinese herbal medicines consist of: 3 kg of Ophiopogon japonicus, 6 kg of Astragalus, 5 kg of Matsutake, 8 kg of Panax notoginseng, 3 kg of Sea buckthorn, 4 kg of Honeysuckle, 8 kg of Atractylodes macrocephala, 13 kg of Chinese yam, 1.5 kg of Achyranthes bidentata and 1.6 kg of Poria cocos.

The second compounding components consist of: 13 kg of beer yeast, 3 kg of linseed oil, 2 kg of cod liver oil, and 1.5 kg of corn oil.

The method for preparing the aforesaid diet for baby dogs consists of following steps:

S1. Mix the high protein materials and the vegetable adhesive evenly, and then, feed them to the mill and grind it for 30 min, and the milling pressure is 0.16 MPa, then, add water till the water content reaches 50 wt %, after that, add multiple vitamins and compound trace elements, stir them evenly, and then, cook them for 30 min, and cool them down, as well as inoculate fermentative bacteria preparation, after that, vacuumize them, and thus, obtain the main component;

S2. Mix the Chinese herbal medicines evenly, and then, feed them to extracting tank, and add water of 2.5 times of the total mass of the Chinese herbal medicines, and then, bring to a boil, after that, cook them in the boiling state for 3 h, and then, filter them, and thus, obtain the first stage filtrate and the filter residue; and then, add water of 1.7 times of the total mass of the filter residue to the filter residue, after bringing to a boil, cook them in the boiling state for 3 h, and then, filter them, and thus, obtain the second stage filtrate; after that, combine these two filtrates, and then, add montmorillonite and fish meal for stirring at the temperature of 82-88° C., and then, concentrate them till the water content reaches 30 wt %, and then sieve them through a 100-mesh sieve after pulverizing by pulverizer, and then, vacuumize them, and thus, obtain the first auxiliary component;

S3. Mix the beer yeast, linseed oil, cod liver oil, and corn oil evenly, and then, vacuumize them, and thus, obtain the second compounding component.

The method for using the aforesaid diet for baby dogs consists of following steps: heat the main component for 15 min at the temperature of 35° C., and stir them evenly during the heating process, after that, add the first auxiliary component and keep stirring for 15 min, and then, add the second compounding component and keep stirring for 10 min, after that, feed them to baby dogs.

### Comparative Example 5

A kind of diet for baby dogs, wherein, the raw materials of which consist of 85 kg of the main components, 10 kg of the first auxiliary components, and 5 kg of the second compounding components.

The main components in terms of mass percent consist of: 85% of high protein material, 7% of vegetable adhesive, 0.15% of multiple vitamins, 0.05% of compound trace elements, and the remain is fermentation preparation.

The high-protein materials consist of: 20 kg of chicken, 8 kg of shrimp meal, 5 kg of eggs, 14 kg of bean pulp; the vegetable adhesive is composed of 4 kg of purple sweet potato flour and 4.5 kg of corn starch; and the fermentation preparation is composed of 0.11 kg of fermentative bacteria preparation and 0.15 kg of proteinase preparation, wherein, the fermentative bacteria preparation consists of: Pichia pastoris, Lactobacillus brucei, Lactobacillus plantarum, Pediococcus acidilactici, and Lactobacillus, and the inoculation amount is 2.5×108 cfu/kg.

The multiple vitamins consist of: vitamin A, vitamin D3, vitamin E, vitamin B1, vitamin B2, nicotinic acid, pantothenic acid, vitamin B6, folic acid, biotin, vitamin B12, and choline chloride; and the compound trace elements consist of: sodium chloride, magnesium lactate, ferrous lactate, copper methionine, zinc lactate, manganese sulfate, calcium iodate, and selenium yeast.

The contents of vitamins and trace elements are as follows:

| | | |
|---|---|---|
| Vitamin A | 5779.35 | IU/kg |
| Vitamin D₃ | 613.22 | IU/kg |
| Vitamin E | 57.38 | IU/kg |
| Vitamin B₁ | 1.23 | mg/kg |
| Vitamin B₂ | 2.55 | mg/kg |
| Nicotinic acid | 12.93 | mg/kg |
| Pantothenic acid | 11.70 | mg/kg |
| Vitamin B₆ | 1.23 | mg/kg |
| Folic acid | 231.17 | μg/kg |
| Biotin | 113.4 | μg/kg |
| Vitamin B₁₂ | 27.87 | μg/kg |
| Choline | 1.34 | mg/kg |
| Sodium chloride | 12.38 | g/kg |
| Magnesium | 450.07 | mg/kg |
| Iron | 67.51 | mg/kg |
| Copper | 8.18 | mg/kg |
| Zinc | 56.26 | mg/kg |
| Manganese | 5.63 | mg/kg |
| Iodine | 1.74 | mg/kg |
| Selenium | 0.96 | mg/kg |

The first auxiliary components in terms of mass percent consist of: 20% of fish meal, 13% of seaweed, and the remain is Chinese herbal medicines; wherein, the Chinese herbal medicines consist of: 3 kg of Ophiopogon japonicus, 6 kg of Astragalus, 5 kg of Matsutake, 8 kg of Panax notoginseng, 3 kg of Sea buckthorn, 4 kg of Honeysuckle, 8 kg of Atractylodes macrocephala, 13 kg of Chinese yam, 1.5 kg of Achyranthes bidentata and 1.6 kg of Poria cocos.

The second compounding components consist of: 13 kg of beer yeast, 3 kg of linseed oil, 2 kg of cod liver oil, and 1.5 kg of corn oil.

The method for preparing the aforesaid diet for baby dogs consists of following steps:

S1. Mix the high protein materials evenly, and add proteinase preparations for enzymatic hydrolysis for 14 h at the temperature of 34° C., and then, heat up to 90° C. and complete hyperthermic treatment for 30 min, after that, feed them to the blender mixer, and then, add vegetable adhesive and stir evenly, after that, feed them to the mill and grind it for 30 min, and the milling pressure is 0.16 MPa, then, add water till the water content reaches 50 wt %, after that, add multiple vitamins and compound trace elements, stir them evenly, and then, cook them for 30 min, and cool them down, as well as inoculate fermentative bacteria preparation, after that, vacuumize them, and thus, obtain the main component;

S2. Mix the Chinese herbal medicines evenly, and then, feed them to extracting tank, and add water of 2.5 times of the total mass of the Chinese herbal medicines, and then, bring to a boil, after that, cook them in the boiling state for 3 h, and then, filter them, and thus, obtain the first stage filtrate and the filter residue; and then, add water of 1.7 times of the total mass of the filter residue to the filter residue, after bringing to a boil, cook them in the boiling state for 3 h, and then, filter them, and thus, obtain the second stage filtrate; after that, combine these two filtrates, and then, add montmorillonite and fish meal for stirring at the temperature of 82-88° C., and then, concentrate them till the water content reaches 30 wt %, and then sieve them through a 100-mesh sieve after pulverizing by pulverizer, and then, vacuumize them, and thus, obtain the first auxiliary component;

S3. Mix the beer yeast, linseed oil, cod liver oil, and corn oil evenly, and then, vacuumize them, and thus, obtain the second compounding component.

The method for using the aforesaid diet for baby dogs consists of following steps: heat the main component for 15 min at the temperature of 35° C., and stir them evenly during the heating process, after that, add the first auxiliary component and keep stirring for 10 min, and then, add the second compounding component and keep stirring for 15 min, after that, feed them to baby dogs.

Conduct a comparative test on the diets obtained in the Embodiment 5 which can enhance the immune function of baby dogs, the wet food used in the Comparative Example 1, and the diets for baby dogs obtained in the Comparative Examples 2-5:

Selecting 60 4-month-old Beagle puppies (the puppies have been dewormed and immunized, and their growth and development, nutritional status, appetite, and body condition are all normal), and randomly dividing them into 6 groups, each with 10 puppies, as well as feeding them in isolation. Using the diet obtained from the Embodiment 5 which can enhance the immune function of baby dogs, the wet food used in the Comparative Example 1, and the diet for baby dogs obtained from the Comparative Examples 2-5 respectively to feed puppies. The daily feeding amount is 200 g, and feed them for 3 times. Puppies are allowed to drink freely, and do physical and technical training for 1 hour in the morning and in the afternoon each day; and observe their mental state, the condition of coat development and feces at any time during the test. The test period is 30 days, and conduct test assessments every 10 days, 20 days, and 30 days respectively. Prior to the 30^{th} day, complete collection of limb venous blood, and collect 1.5-3 mL of blood from each puppy every time, and then, the blood samples collected are stored in sodium citrate negative pressure storage vessels. Take one sample to be treated with hemolysin solution, and then, put it into automatic blood cell analyzer for testing; after that, take another sample to put into the centrifugal machine, and then, centrifuge it at 4000 r/min for 30 min to separate the serum, then, take the upper layer of serum and transfer it into each test cup as well as place it in the refrigerator at the temperature of 4° C. for standby application, and complete biochemical indexes test. The results are as follows:

| | Ter- | Apparent | | | |
|---|---|---|---|---|---|
| | minal | Di- | | | |
| | Body | gestibility | | | Feces |
| | Weight, | of Crude | Mental | Coat | Classi- |
| | Kg | Protein % | State | Development | fication |
| Embodiment | 7.7 | 82.15 | Good | The coat | 2 |
| 5 | | | | is very | |
| | | | | smooth with | |
| | | | | less hair loss. | |
| Comparative | 5.3 | 64.27 | Good | The coat is | 3 |
| Example 1 | | | | not smooth, | |
| | | | | the glossiness | |
| | | | | is average, | |
| | | | | and there is | |
| | | | | more hair | |
| | | | | loss. | |
| Comparative | 6.0 | 74.52 | Average | The coat | 3.5 |
| Example 2 | | | | is smooth | |
| | | | | with more | |
| | | | | hair loss. | |
| Comparative | 5.7 | 72.38 | Poor | The coat is | 1.5 |
| Example 3 | | | | not smooth, | |
| | | | | the glossiness | |
| | | | | is poor, and | |
| | | | | there is more | |
| | | | | hair loss. | |
| Comparative | 6.3 | 69.35 | Good | The coat | 2.5 |
| Example 4 | | | | is smooth | |
| | | | | with more | |
| | | | | hair loss. | |
| Comparative | 6.8 | 78.39 | Average | The coat | 3.5 |
| Example 5 | | | | is very | |
| | | | | smooth with | |
| | | | | less hair loss. | |

The measurement method of the aforesaid apparent digestibility: prior to the experiment, take 500 g of each group of feedstuffs, mix them thoroughly, and then, pulverize them after air drying, after that, sieve them through a 40-mesh sieve, and thus, produce analysis samples; 3 days before the end of each experimental stage, collect about 50 g of feces evenly from each dog cage in the morning and in the evening, and mix them. Collect feces for 3 consecutive days, and mix them thoroughly upon taking cage as a unit, and preserve them after freezing. After that, measure the contents of acid-insoluble ash and crude protein in the feedstuffs and feces respectively, and thus, calculate the apparent digestibility.

$$Apparent Digestibility of Crude Protein = 100 \times ( 1 - \frac{{N}_{f}}{{N}_{d}} \times \frac{{M}_{d}}{{M}_{f}} )$$

Wherein, Nf represents the content of crude protein in the feces, Nd represents the content of crude protein in the feedstuff, Md represents the content of acid-insoluble ash in the feedstuff, and Mf represents the content of acid-insoluble ash in the feces.

The aforesaid feces classifications are as follows:

| Feces | |
|---|---|
| Classifications | Description |
| Class 1 | Dry, hard, and cataclastic. |
| Class 1.5 | Dry and hard. |
| Class 2 | The feces are well formed and would not leave a mark |
| | when picked up. |
| Class 2.5 | The feces are well formed but would leave a mark when |
| | picked up |
| Class 3 | The feces contain water with a little shapeless. |
| Class 3.5 | The feces are watery and almost out of shape. |
| Class 4 | The feces are hardly to form a shape. |
| Class 4.5 | Suffer from diarrhea, but a little part can form a shape. |
| Class 5 | Suffer from watery diarrhea. |

It can be seen from the above table that the digestibility of crude protein of the present invention is high, and thus, improves the health status of the coat of baby dogs, as well as significantly improve the gastrointestinal health of dogs.

The blood cell tests are as follows:

| | WBC, 10⁹/L | RBC, 10⁹/L | LY, 10⁹/L |
|---|---|---|---|
| Prior to the | 8.33 | 4.90 | 1.11 |
| Experiment | | | |
| Embodiment 5 | 13.02 | 7.54 | 3.20 |
| Comparative | 8.40 | 4.45 | 1.51 |
| Example 1 | | | |
| Comparative | 9.23 | 5.07 | 1.70 |
| Example 2 | | | |
| Comparative | 9.98 | 5.83 | 2.25 |
| Example 3 | | | |
| Comparative | 11.48 | 7.01 | 2.99 |
| Example 4 | | | |
| Comparative | 10.52 | 6.45 | 2.65 |
| Example 5 | | | |

The results of serum biochemical tests are as follows:

| | IgG, | IgM, | IgA, | C3, | C4, | SOD, |
|---|---|---|---|---|---|---|
| | g/L | g/L | g/L | mL/L | mL/L | U/L |
| Prior to the | 25.22 | 15.99 | 23.21 | 5.25 | 5.60 | 100.50 |
| Experiment | | | | | | |
| Embodiment | 39.27 | 27.45 | 52.02 | 17.93 | 8.14 | 134.68 |
| 5 | | | | | | |
| Comparative | 25.38 | 16.03 | 23.11 | 5.03 | 5.84 | 99.84 |
| Example 1 | | | | | | |
| Comparative | 27.46 | 18.31 | 30.53 | 7.21 | 5.93 | 103.59 |
| Example 2 | | | | | | |
| Comparative | 29.37 | 21.93 | 38.70 | 10.61 | 6.56 | 116.73 |
| Example 3 | | | | | | |
| Comparative | 36.36 | 25.83 | 49.47 | 15.33 | 7.61 | 129.57 |
| Example 4 | | | | | | |
| Comparative | 31.44 | 23.15 | 44.29 | 12.52 | 6.66 | 123.38 |
| Example 5 | | | | | | |

The contents of white blood cell count (WBC), red blood cell count (RBC), lymphocyte count (LY), immune globulins (IgG, IgM, IgA), and complements (C3, C4) are important indicators that reflect the immune level of body. Meanwhile, superoxide dismutase (SOD) is a kind of metal enzyme widely existed in flora and fauna as well as microorganisms, which can catalyze the disproportionation reaction of superoxide free radicals (O2-) in organisms. It is a natural scavenger existed in the body, which plays an important role in the self-protection system and immune system of animals and plants.

It can be seen from the above table that the present invention improves various immune indices of beagle puppies through organic combination of a variety of approaches, especially the effects on complements of C3, C4, IgA, IgG, IgM are obvious, in addition, it can improve the number of lymphocytes and white blood cells of body, and thus, has a certain impact on the content of red blood cells, and indirectly improves the immunity of the body, so that stimulates the body immunity of beagle puppies in many aspects and enhances the body immunity of beagle puppies; meanwhile, the present invention can stimulate the SOD activity in the body of beagle puppies, and thus, further improves the immunity and disease resistance of beagles.

The aforesaid is only preferred specific embodiments of the present invention, however, the protection scope of the present invention is not limited to this extent. Any equivalent replacements or changes made by those skilled in the prior art according to the technical scheme and the inventive concept of the present invention and within the technical scope disclosed by the present invention shall fall within the protection scope of the present invention.

1. A kind of diet capable of enhancing the immune function of baby dogs, characterized in that its raw materials consist of a main component, a first auxiliary component, and a second compounding component;
the main components in terms of mass percent consist of: 80-90% of high protein material, 5-10% of vegetable adhesive, 0.1-0.2% of multiple vitamins, 0.01-0.1% of compound trace elements, and the remain is fermentation preparation;
the first auxiliary components in terms of mass percent consist of: 20-40% of montmorillonite, 10-30% of fish meal, 10-16% of seaweed, and the remain is Chinese herbal medicines;
the second compounding components consist of: beer yeast, linseed oil, cod liver oil, and corn oil.

2. The said diet capable of enhancing the immune function of baby dogs according to claim 1, characterized in that the mass ratio among the main components, the first auxiliary components, and the second compounding components is 80-90:8-12:4-6.

3. The said diet capable of enhancing the immune function of baby dogs according to claim 1, characterized in that the high-protein materials consist of: chicken, shrimp meal, eggs, bean pulp; and the mass ratio among chicken, shrimp meal, eggs, and bean pulp is 15-25:4-12: 2-8:10-18.

4. The said diet capable of enhancing the immune function of baby dogs according to claim 1, characterized in that the vegetable adhesive is composed of purple sweet potato flour and corn starch, and the mass ratio between purple sweet potato flour and corn starch is 2-6:3-6.

5. The said diet capable of enhancing the immune function of baby dogs according to claim 1, characterized in that the fermentation preparation is composed of fermentative bacteria preparation and proteinase preparation, and the mass ratio between the fermentative bacteria preparation and the proteinase preparation is 0.1-0.12:0.1-0.2.

6. The said diet capable of enhancing the immune function of baby dogs according to claim 5, characterized in that the fermentative bacteria preparation consists of: Pichia pastoris, Lactobacillus brucei, Lactobacillus plantarum, Pediococcus acidilactici, and Lactobacillus, and the inoculation amount is 2.5×108 cfu/kg.

7. The said diet capable of enhancing the immune function of baby dogs according to claim 1, characterized in that the Chinese herbal medicines in terms of part by mass consist of: 1-6 parts of Ophiopogon japonicus, 2-10 parts of Astragalus, 2-8 parts of Matsutake, 3-12 parts of Panax notoginseng, 1-5 parts of Sea buckthorn, 2-6 parts of Honeysuckle, 4-12 parts of Atractylodes macrocephala, 10-16 parts of Chinese yam, 1-2 parts of Achyranthes bidentata and 1-2 parts of Poria cocos.

8. The said diet capable of enhancing the immune function of baby dogs according to claim 1, characterized in that the mass ratio among beer yeast, linseed oil, cod liver oil, and corn oil is 10-16:2-4:1-3:1-2.

9. A kind of method for preparing the said diet capable of enhancing the immune function of baby dogs according to claim 1, characterized in that consisting of following steps:
S1. Mix the high protein materials evenly, and add proteinase preparations for enzymatic hydrolysis, after hyperthermic treatment, add vegetable adhesive, and then stir evenly, after that, mill them, and then add water, and multiple vitamins as well as compound trace elements for stirring evenly, and then, cook them and cool them down, as well as inoculate fermentative bacteria preparation, after that, vacuumize them, and thus, obtain the main component;
S2. Mix the Chinese herbal medicines evenly, and add water, and then, bring to a boil, after that, cook them in the boiling state, and then, filter them, and thus, obtain the first stage filtrate and the filter residue; and then, add water to the filter residue, after bringing to a boil, cook them in the boiling state, and then, filter them, and thus, obtain the second stage filtrate; after that, combine these two filtrates, and then, add montmorillonite and fish meal for stirring and concentrating, after that, pulverize them by pulverizer and sieve them, and then, vacuumize them, and thus, obtain the first auxiliary component;
S3. Mix the beer yeast, linseed oil, cod liver oil, and corn oil evenly, and then, vacuumize them, and thus, obtain the second compounding component.

10. A kind of method for using the said diet capable of enhancing the immune function of baby dogs according to claim 1, characterized in that consisting of following steps: heat the main component, and stir it evenly during the heating process, after that, add the first auxiliary component and keep stirring, and then, add the second compounding component and keep stirring, after that, feed them to baby dogs.
